(19) United States
(12) Patent Application Publication
(10) Pub. No.: US 2010/0048931 A1
(43) Pub. Date: Feb. 25, 2010

(54) OXIDATION-STABLE CARBOXYLIC ESTERS AND USE THEREOF

(76) Inventors: Alfred Westfechtel, Hilden (DE); Matthias Hof, Duisburg (DE)

Correspondence Address: SMITH MOORE LEATHERWOOD LLP, P.O. BOX 21927, GREENSBORO, NC, 27420, US

(21) Appl. No.: 12/524,826

(22) PCT Filed: Feb. 1, 2008
(86) PCT No.: PCT/EP2008/000821

§ 371 Date: Oct. 20, 2009

(30) Foreign Application Priority Data
Feb. 2, 2007 (EP) 07002256.1

(51) Int. Cl. C07C 57/02

(52) U.S. Cl. 554/223

(57) ABSTRACT

Carboxylic acid esters which contain as the alcohol component trimethylolpropane and as the acid component a mixture of (i) linear saturated fatty acids having 8 to 18 C atoms and (ii) linear mono- or polyunsaturated fatty acids having 12 to 22 C atoms, wherein the molar ratio of (i):(ii) is in the range of from 3:1 to 1:3, show a good stability to oxidation and are suitable as base oils for hydraulic oils.

The invention relates to selected carboxylic acid esters and the use thereof as a constituent of hydraulic oils or in lubricants.

Hydraulic systems are employed in a diversity of technical equipment, for example automobiles, heavy goods vehicles, cranes, trains and other means of transport, but also in agricultural equipment, ships and in industrial plants and in the railway sector. The hydraulic systems as a general rule contain a hydraulic liquid, which traditionally contains petrochemical liquids or esters or other oleochemicals as the base liquid. The latter are increasingly preferred because of their biodegradability.

In this connection, in particular polyol esters of fatty acids, preferably unsaturated fatty acids, are known from the prior art as suitable base liquids for hydraulic oils. Thus, WO 97/39086 discloses esters which are obtained by reaction of polyols, including also trimethylolpropane, with mixtures of fatty acids, where the ratio of short-chain fatty acids to the long-chain fatty acids must be in the range of from 2:1 to 1:20. The specification discloses that the esters claimed are said to have advantageous technical properties in particular because of their tolerance to low temperatures. Nevertheless, a prerequisite of WO 97/39086 is mixtures of fatty acids wherein the short-chain content is chosen solely from the group of C5-C12 fatty acids, in the examples exclusively the C8-C10 fatty acids. DE 101 15 829 A1 describes oxidation-stable polyol esters which can be prepared by employing technical-grade oleic acid as the acid component.

In addition to the stability to low temperatures, ester systems and hydraulic liquids must nevertheless also have a high stability to oxidation, which is relevant in particular when the hydraulic oil is exposed to high temperatures in the presence of atmospheric oxygen. In order to achieve this, according to the prior art antioxidants are admixed to hydraulic oils gas conventional additives. The so-called modified, dry “turbine oxidation stability test”, called dry TOST for short according to DIN51587, which tests the stability of test oils on ageing at 95° C. in the presence of oxygen, is decisive in this case. Conventional systems already reach the critical limit value of 2.0 mg of KOH/g of test oil here after 170 to about 300 hours. However, since an increasingly higher stability to oxidation is required, there is a constant need on the part of industry to be able to provide oils which are more stable to oxidation. The attempt to achieve the desired high stability to oxidation by addition of further antioxidants has so far not yet been successful.

It was therefore the object of the present invention to provide oxidation-stable liquids for use in hydraulic oils which also meet current requirements with respect to their biodegradability. It has been found, surprisingly, that selected polyol esters meet the above requirements.

The present application therefore provides synthetic carboxylic acid esters which contain as the alcohol component trimethylolpropane and as the acid component a mixture of (i) linear saturated fatty acids having 8 to 18 C atoms and (ii) linear mono- or polyunsaturated fatty acids having 12 to 22 C atoms, wherein the molar ratio of (i):(ii) is in the range of from 3:1 to 1:3 and preferably in the range of from 2:1 to 1:3 and particularly preferably in the range of from 1:1 to 1:3.

The synthetic esters according to the invention are prepared in a manner known per se by reaction of trimethylolpropane with mixtures of saturated and unsaturated fatty acids in the presence of suitable catalysts and at elevated temperature. The reaction products can then be removed from the reaction mixture by distillation. It may be advantageous to carry out the reaction under an inert gas atmosphere, preferably nitrogen.

An essential technical feature of the preparation of the esters according to the invention is the molar ratio of the fatty acid mixture. Only in the narrow range claimed of from 3:1 to 1:3 or preferably 2:1 to 1:3 and particularly preferably in the range of from 1:1 to 1:3 are the desired esters obtained. A further preferred range for the molar ratio of (i) to (ii) is 2:1 to 1:2, and here preferably 1:1 to 1:2.

Saturated linear fatty acids which can be used are those having 8 to 18, preferably 8 to 16 and in particular 8 to 14 C atoms. Acids which are concretely and preferably possible are: octanoic acid, pelargonic acid, nonanoic acid, decanoic acid, undecanoic acid, dodecanoic acid, tridecanoic acid, tetradecanoic acid, pentadecanoic acid, palmitic acid, margaric acid, stearic acid and nonadecanoic acid and any desired mixtures of these acids with one another. Mixtures of C8 to C14 acids are preferably employed for synthesis of the esters according to the invention. Particularly preferred linear saturated fatty acids (i) are those fatty acid mixtures which contain fatty acids of chain length C-12 to C-18 and preferably C-12 to C-14 to the extent of more than 80 wt. %, preferably more than 85 wt. % and in particular more than 90 wt. %. Preferred fatty acid mixtures of type (i) are free from fatty acids of chain length C-18. Acid mixtures of octanoic acid and decanoic acid in the weight ratio of 55:45 are also not included as component (i). These are disclosed concretely in WO 97/39086 in the examples.

Oleic acid is chosen as an unsaturated fatty acid. This can also be employed in technical-grade quality, but a pure oleic acid is preferred. Oleic acid is essentially obtained from its natural occurrence in plant and animal fats and oils. For this, the triglycerides are first subjected to cleavage under pressure and the resulting mixtures of the various fatty acids are separated by the process of phase inversion into a saturated content (stearin) and an unsaturated content largely consisting of oleic acid (olein). A purified oleic acid is preferably used. However e.g. a technical-grade oleic acid such as is obtained e.g. by cleavage under pressure and subsequent separation by phase inversion is less suitable. Technical-grade oleic acid typically contains only approx. 70 wt. % of the monounsaturated oleic acid, the remaining 30 wt. % also falling to polyunsaturated acids, and unsaturated acids, partly C-18 and C-16. However, the present invention advantageously and therefore preferably makes use of pure oleic acid qualities which contain more than 70%, preferably more than 80% and in particular more than 90% of oleic acid.

However, those fatty acid mixtures such as are disclosed concretely in DE 101 15 829 A1 cited above, in column 2, line 60 to column 3, line 9, and in the embodiment example of this specification are excluded.

Those esters in which the molar ratio between the acid components (as the acids of type (i) and (ii) together) and trimethylolpropane is in the range of from 5:1 to 1:1, preferably 2:1 to 1:1 and particularly preferably 1.5:1 to 1:1, the ratios during the synthesis—that is to say the molar ratios of the starting compounds—being taken into account here, are particularly preferred.

A further aspect of the present invention relates to the use of the esters described as constituents of hydraulic oil, and in particular as a foundation or base oil therein. Such hydraulic oils can then preferably contain up to 95 wt. % of the esters, but amounts of between 25 and 85 wt. % are preferred. In addition to the base oil, these oils as a rule also contain additives, preferably in amounts of from 5 to 25 wt. %. The additives are the classes known in principle to the person skilled in the art, namely antioxidants, extreme pressure (EP) or anti-wear (AW) additives, corrosion inhibitors, demulsifiers and/or defoamers. They can furthermore also contain nonferrous metal deactivators.

The hydraulic oil contains the additives in conventional amounts, but in amounts in total of max. 10 wt. %, preferably 1 to 3 wt. %, based on the total weight of the hydraulic oil. The hydraulic oils according to the invention contain the EP/AW additives here in amounts of from preferably 0.2 to 2.0 wt. %, antioxidants in the range of from 0.2 to 1.0 wt. %, corrosion protection additives in the range of from 0.05 to 0.2 wt. %, nonferrous metal deactivators in the range of from 0.05 to 0.5 and antifoam additives or defoamers in the range of from 0.005 to 0.04%.

Such hydraulic oils are formulated by mixing the base liquid with the additives, optionally at elevated temperature. The esters of the present application can also and preferably be used as a constituent of lubricants.

### EXAMPLES

1. Preparation of the Esters According to the Invention

100 g of a mixture of saturated linear fatty acids of the C chain cut C8-C14 (0.5 mol) were mixed with 280 g of oleic acid (1.0 mol) and 0.45 g of a catalyst (FASCAT® 2001, Arkema). This mixture was heated together with 74 g of trimethylolpropane (=TMP) (0.55 mol) at 240° C. for several hours. The fatty acid mixture of the short-chain linear saturated fatty acids (i) had the following composition (figures in wt. %): 7% of C-8, 8% of C-10, 62% of C-12 and 19% of C-14 fatty acids. A technical-grade quality was employed as the oleic acid. The water was then removed by distillation. The crude reaction product was cooled and filtered. The yield was 99% of theory.

### 2. Preparation of the Comparison Esters

Analogously to the procedure as described above, the comparison esters were also prepared, but other weight ratios were chosen. Details are to be found in the following table. In all cases TMP was the alcohol component.

TABLE 1

| | | | | Cloud | Pour | Dyn. | | | |
|---|---|---|---|---|---|---|---|---|---|
| | Fatty | Wt. | Mol. | point | point | viscosity | | | |
| No. | acid(s) | ratio | ratio | [° C.] | [° C.] | [cps] | SN | AN | OHN |
| 1 | C8-C14 | 100 | 100 | −5 | −5 | 34 | 243 | 3.4 | 27 |
| 2 | Oleic acid | 100 | 100 | −30 | −40 | 45 | 190 | 0.7 | 15 |
| 3 | C8-C14/ | 78:22 | 1:0.5 | −10 | −11 | 40 | 229 | 3.4 | 26 |
| | oleic acid | | | | | | | | |
| 4 | C8-C14/ | 42:48 | 1:1 | −20 | −22 | 42 | 206 | 3.3 | 23 |
| | oleic acid | | | | | | | | |
| 5 | C8-C14/ | 26:74 | 0.5:1 | −24 | −28 | 40 | 198 | 0.95 | 19 |
| | oleic acid | | | | | | | | |

It can be seen that the TMP esters 3 to 5 chosen according to the invention have the best low temperature properties compared with esters 1 and 2.

### 3. Use Properties

To test the use properties of the esters, these were tested in accordance with DIN 51587 (so-called DRY TOST). Esters 3 to 5 according to the invention according to Table 1 and the ester of oleic acid with TMP, no. 2, were each investigated with an additive package containing, inter alia, antioxidants, metal deactivators, defoamers etc.

The acid numbers as a function of time were measured. Results of the difference in acid numbers are reproduced in the following table. The investigation was ended as soon as the difference in the acid number was 2 or more.

TABLE 2

| | Fatty | | | | |
|---|---|---|---|---|---|
| No. | acid(s) | Start | 168 h | 336 h | 504 h |
| 2 | Oleic acid | 0 | 11.3 | — | — |
| 5 | C8-C14/ | 0 | 0.4 | 0.7 | 10.6 |
| | oleic acid | | | | |

TMP ester no. 5 according to the invention has an improved stability to oxidation compared with a pure oleic acid ester.

1. A carboxylic acid ester which contains as the alcohol component trimethylolpropane and as the acid component a mixture of
(i) linear fatty acids having 8 to 18 C atoms, wherein more than 80 wt-% of the fatty acid mixture have fatty acids of a chain length C-12 to C-18 and
(ii) linear mono- or polyunsaturated fatty acids having 12 to 22 C atoms, characterized in that,
the molar ratio of (i):(ii) is in the range of from about 3:1 to about 1:3.

2. The carboxylic acid ester according to claim 1, characterized in that the molar ratio between the acid component and trimethylolpropane is in the range of from about 5:1 to about 1:1.

3. The carboxylic acid ester according to claim 1 characterized in that fatty acids having 8 to 14 C atoms are chosen as acid component (i).

4. The carboxylic acid ester according to claim 1 characterized in that oleic acid is chosen as acid component (ii).

5. The carboxylic acid ester according to claim 1 characterized in that the linear saturated fatty acids (i) are used in fatty acid mixtures, which contain more than about 80% fatty acids having a chain length of C-12 to C-14.

6. The carboxylic acid ester according to claim 5, characterized in that oleic acid is used as the unsaturated fatty acid (ii).

7. A use of carboxylic acid esters, which contain as the alcohol component trimethylolpropane and as the acid component a mixture of (i) linear saturated fatty acids having 8 to 16 C-atoms and (ii) linear mono- or polyunsaturated fatty acids having 12 to 22 C-atoms, wherein the molar ratio of (i):(ii) is in the range of from 3:1 to 1:3.

8. The use according to claim 7, characterized in that the molar ratio between the acid component and trimethylolpropane is in the range of from about 5:1 to about 1:1.

9. The use according to claim 7 characterized in that as the acid component (i) fatty acids are used having from about 8 to about 16.

10. The use according to claim 7 characterized in that as the acid component (ii) oleic acid is used.

11. The use according to claim 7 characterized in that the linear saturated fatty acids (i) are used in fatty acid mixtures, which contain more than 80% of fatty acids with a chain length of C-12 and C-14.

12. The use according to claim 11, characterized in that, as unsaturated fatty acid (ii) oleic acid is used.

13. The use of carboxylic acid esters according to the definition in claim 7 as a lubricant.

14. A hydraulic oil, containing a carboxylic acid ester according to the definition in claim 7 in a quantity up to about 95 wt-%.

15. The carboxylic acid ester according to claim 1 wherein the molar ratio of (i):(ii) is in the range of from about 1:1 to about 1:3.

16. The carboxylic acid ester according to claim 1 wherein the molar ratio between the acid component and trimethylolpropane is in the range of from about 2:1 to about 1:1.

17. The carboxylic acid ester according to claim 1 characterized in that the linear saturated fatty acids (i) are used in fatty acid mixtures, which contain more than about 90% fatty acids having a chain length of C-12 to C-14.

18. A use of carboxylic acid esters, which contain as the alcohol component trimethylolpropane and as the acid component a mixture of (i) linear saturated fatty acids having 8 to 16 C-atoms and (ii) linear mono- or polyunsaturated fatty acids having 12 to 22 C-atoms, wherein the molar ratio of (i):(ii) is in the range of from about 1:1 to about 1:3, as base liquid in hydraulic oils or in lubricants.

19. The use according to claim 7, characterized in that the molar ratio between the acid component and trimethylolpropane is in the range of from about 1.5:1 to about 1:1.

20. The use according to claim 7 characterized in that as the acid component (i) fatty acids are used having from about 8 to about 14 C-atoms.

21. The use according to claim 7 characterized in that the linear saturated fatty acids (i) are used in fatty acid mixtures, which contain more than about 90% of fatty acids with a chain length of C-12 and C-14.

22. The hydraulic oil, containing a carboxylic acid ester according to the definition in claim 7 in a quantity of from about 5 to about 25 wt-%.
